(19) United States
(12) Patent Application Publication
(10) Pub. No.: US 2024/0279681 A1
(43) Pub. Date: Aug. 22, 2024

(54) METHOD FOR PURIFICATION OF GMP-GRADE RETROVIRAL VECTOR AND APPLICATION THEREOF

(71) Applicant: CARBIOGENE THERAPEUTICS CO., LTD., Hangzhou (CN)

(72) Inventors: Jiangao ZHU, Hangzhou (CN); Jiajia LI, Hangzhou (CN); Wenjun YANG, Hangzhou (CN)

(21) Appl. No.: 18/571,292

(22) PCT Filed: Nov. 30, 2021
(86) PCT No.: PCT/CN2021/134470

§ 371 Date: Dec. 18, 2023

(30) Foreign Application Priority Data
Jun. 18, 2021 (CN) 202110676917.3

(51) Int. Cl. C12N 15/86

(52) U.S. Cl. CPC C12N 15/86; C12N 2740/10043; C12N 2740/10051

(57) ABSTRACT

The invention discloses a method for purifying a retrovirus (retroviral vector), comprising the following steps: A), microfiltrating a cell culture supernatant containing a retrovirus to remove cell debris from the cell culture supernatant and obtain a micro-filtered virus solution; B), treating the micro-filtered virus solution with a nuclease to degrade host DNA residues into small DNA fragments, and obtain an enzyme-digested virus solution; C), ultrafiltering the enzyme-digested virus solution such that the retrovirus is retained in a retentate, collecting the retentate, and obtaining an ultrafiltrated virus solution; and D), performing low speed centrifugation on the ultrafiltered virus solution to precipitate the retrovirus, collecting the precipitate to obtain unsterilized virus. The retrovirus product obtained by this method ensures the purity of the retrovirus solution, and at the same time, the titer of the concentrated virus solution reaches 10⁷ IP/ml, which meets the downstream process requirements.

### TECHNICAL FIELD

The present invention relates to the field of biotechnology, specifically to a method for purification of GMP-grade retroviral vector and application thereof, in particular to the purification method of GMP-grade retroviral vector that can be applied to chimeric antigen receptor T-cell therapy and application thereof.

### BACKGROUND OF THE INVENTION

Gene therapy has been shown great therapeutic application potential for a variety of diseases treatment, and is also considered to be the ultimate means of next-generation clinical treatment. Gene therapy refers to the introduction of foreign DNA fragments into target cells to perform targeted intervention on defective and abnormal genes by correcting, repairing, replacing, compensating or silencing, in order to restore normal gene functions and finally achieve the goal of treatment or even complete cure. Retrovirus-derived vectors have become one of the most widely used vectors in clinical trials of gene therapy. Currently, the preparation of retroviral vectors still faces an enormous challenge. There are two main ways to deliver clinical application of viral vectors: This first one is called ex vivo, which refers to infect target cells in vitro, culture and amplify the target cells imported with exogenous genes in vitro and then infuse back into the human body, such as CAR-T cell therapy; the other one is called in vivo, which refers to directly infect the target cells and tissues in the body with purified viral vectors, such as oncolytic virotherapy. For the former, the clinically required viral activity titer is generally in the order of 10⁷ IP/mL; for the latter, the viral activity titer is even required to reach the order of 10⁹ IP/mL. However, the traditional method of obtaining retrovirus (retroviral vector) liquid by collecting the supernatant of stable production strains cannot meet this requirement. With the increasing demand for clinical-grade high-purity retrovirus (retroviral vector), there is an urgent need to develop new manufacturing processes for the preparation and purification of retrovirus that are efficient and suitable for large-scale production.

The general manufacturing process for retrovirus (retrovirus vector) is to be started with transfecting a cell line expressing retrovirus gag, pol and env genes with a plasmid containing the target gene, and then obtaining stable production strains containing target genes through multiple rounds of subclonal screening to secrete recombinant retrovirus. Subsequently, the stable strains are expanded and cultured when needed, and the supernatant is collected to obtain a crude extract of retrovirus for subsequent purification steps. Compared with adenovirus and lentivirus, retroviruses are not very stable and more sensitive to shear force. Therefore, higher requirements are needed in the purification manufacturing process, especially for the process control of tangential flow ultrafiltration. In addition, traditional ultracentrifugation methods are not suitable for the purification of retrovirus due to the following limitations: expensive centrifuges are required, time consuming, and hard to scale-up. Therefore, the purification of a retrovirus (retroviral vector) is a common problem faced by the industry. There is no complete solution for preparation and purification method of GMP (Good Manufacturing Practice of Medical Products) grade retrovirus suitable for clinical treatment.

### SUMMARY OF THE INVENTION

The present invention provides a method for purifying a retrovirus (retroviral vector), which comprises the following steps:
A), performing microfiltration of a cell culture supernatant containing a retrovirus (retroviral vector) to remove cell debris to obtain a microfiltrated virus solution;
B), performing treatment on the micro-filtered virus solution with a nuclease to degrade host DNA residues into small DNA fragments to obtain an enzyme-digested virus solution;
C), performing ultrafiltration of the enzyme-digested virus solution to allow the retrovirus (retrovirus vector) to be remained in a retentate, collect the retentate, and obtain the ultrafiltered virus solution; and
D), performing a low speed centrifugation of the ultrafiltered virus solution, and collecting the precipitate to obtain an unsterilized virus.

Further, the method comprises a step of filtering and sterilizing the unsterilized virus to obtain a purified retrovirus (retroviral vector).

Further, a PVDF membrane filter with a pore size of 0.22 μm is used to filter and sterilize the above-mentioned viruses.

Further, in step A), in the microfiltration, the membrane pore size (micromembrane pore size) of the semipermeable membrane is 0.45 μm to 0.75 μm, and/or the membrane material of the semipermeable membrane is modified polyethersulfone.

In the microfiltration, the membrane pore size (micromembrane pore size) is 0.45 μm to 0.65 μm, or 0.65 μm or 0.45 μm.

In the microfiltration, the flow rate of cell culture supernatant is 89 mL·min⁻¹ to 133.5 mL·min⁻¹, or 89 mL·min⁻¹ or 133.5 mL·min⁻¹.

In the microfiltration, the shear rate of the microfiltration is 2000 s⁻¹ to 3000 s⁻¹, or 2000 s⁻¹ or 3000 s⁻¹.

The microfiltration can specifically be any of the following:
MF1, the membrane pore size (micromembrane pore size) of the semipermeable membrane used is 0.65 μm, the flow rate of cell culture supernatant is 89 mL·min⁻¹, and the shear rate of the microfiltration is 2000 s⁻¹;
MF2, the membrane pore size (micromembrane pore size) of the semipermeable membrane used is 0.65 μm, the flow rate of cell culture supernatant is 133.5 mL·min⁻¹, and the shear rate of the microfiltration is 3000 s⁻¹;
MF3, the membrane pore size (micromembrane pore size) of the semipermeable membrane used is 0.45 μm, the flow rate of cell culture supernatant is 89 mL·min⁻¹, and the shear rate of the microfiltration is 2000 s⁻¹.

Further, in step B), in the nuclease treatment, the nuclease concentration in the reaction system is 1 to 500 U·mL⁻¹, and/or the reaction is performed at 2° ° C. to 8° C. for 8 h to 24 h.

In the nuclease treatment, the nuclease concentration is 25 U·mL⁻¹, 50 U·mL⁻¹ or 100 U·mL⁻¹;

In the nuclease treatment, the nuclease treatment time is 8 h, 16 h or 24 h;

In the nuclease treatment, the nuclease treatment temperature is 4° C. or 37° C.;

The nuclease treatment can specifically be any of the following:
b1), the concentration of nuclease in the reaction system is 100 U·mL⁻¹, and/or the reaction is performed at 4° C. for 16 h;
b2), the concentration of nuclease in the reaction system is 100 U·mL⁻¹, and/or the reaction is performed at 4° C. for 24 h;
b3), the concentration of nuclease in the reaction system is 50 U·mL⁻¹, and/or the reaction is performed at 4° C. for 24 h.

Further, in step C), the molecular weight cutoff of the ultrafiltration membrane in the ultrafiltration concentration step is 350 KD, 500 KD or 750 KD, preferably 750 KD.

Further, in step C), ultrafiltration and concentration are performed twice as follows:
c1) first, a hollow fiber column with an inner diameter of 0.5 mm and a membrane area of 0.16 m² is used; the shear rate is controlled to 2000 s⁻¹ to ultrafilter and concentrate the enzyme-digested virus solution to obtain a 5-fold concentrated primary ultrafiltration-concentrated virus solution; the flow rate of viral liquid in c1) is 370 mL·min⁻¹;
c2) then a hollow fiber column with an inner diameter of 0.5 mm, a membrane area of 115 cm² is used and a shear rate is controlled to 2000 s⁻¹ to ultrafilter and concentrate the primary ultrafiltration-concentrated virus solution obtained in c1) to obtain a secondary] ultrafiltration-concentrated virus solution by 5-10 times the volume of virus solution in c1); the flow rate of the virus solution in c2 is 53 mL·min⁻¹.

Further, the processing conditions of low speed centrifugation in step D) are: centrifugal force of 4000 g to 10000 g, centrifugation at 4° C. for 4 h to 24 h; preferably, centrifugation at 6000 g, 4° C. for 16 h;
in the low speed centrifugation, the centrifugal force is 4000 g to 8000 g, or 4000 g, 6000 g or 8000 g;
in the low speed centrifugation, the centrifugation time is 4 h to 24 h, or 4 h, 8 h, 16 h or 24 h;
the low speed centrifugal treatment process is specifically any of the following:
d1) centrifuging at 6000 g, 4° ° C. for 16 h;
d2) centrifuging at 6000 g, 4° C. for 8 h.

The present invention provides a retrovirus (retroviral vector) obtained by the above method.

The present invention provides a product (for example, a drug or a vaccine) containing the above-mentioned retrovirus (retroviral vector).

The present invention also provides applications of the retrovirus (retroviral vector) in the preparation of gene therapy products and/or cell therapy products and/or immunotherapy products.

Further, the gene therapy product is an ex vivo gene therapy product, such as a CAR-T cell therapy product.

Compared with the existing technology, the beneficial effects of the present invention are as follows:

1. An innovative combination of purification processes is used to more effectively remove impurities in a retrovirus (retroviral vector) and improve the purity of a retrovirus (retroviral vector). The crude extract of a retrovirus (retroviral vector) is the culture supernatant of stable strains and contains a variety of impurities, including host DNA residues (HCD), host protein residues (HCP), and bovine serum albumin residues (BSA), etc. These impurities may be brought into the human body during clinical application, causing uncontrollable risks to patients, including allergic reactions, tumorigenicity, etc. For this reason, the Pharmacopoeia of the People's Republic of China has a clear upper limit on the residual amounts of impurities. For example, for vaccines, HCD is generally not higher than 10 ng/100 μg protein, BSA is not higher than 50 ng/dose, and HCP is generally not higher than 0.1% of total protein content. As for a retrovirus (retroviral vector), which is intermediate product for ex vivo gene therapy, although there are no clear regulations in current policies and regulations, excessive impurity residues are difficult to remove in downstream processes. Therefore, it is necessary to minimize impurities as possible during the purification of retrovirus to avoid bringing them into downstream processes. The present invent includes microfiltration, ultrafiltration, nuclease treatment, and low speed centrifugation to achieve good removal effects on large fragments, small molecules, and nucleic acid impurities. After the above steps, the host HCD, HCP, BSA and other impurities in retrovirus crude extract can be effectively removed. This process is easy to scale up and meets GMP requirements. The purified retrovirus was tested as BSA<200 ng/ml, HCP<1 μg/ml, and HCD<100 ng/ml. This viral solution has been validated to enter downstream processes of ex vivo gene therapy, such as CAR-T or CAR-NK cell preparation.

2. The present invention maximize the biological activity of retrovirus (retroviral vector) and increase the activity titer. The tangential flow microfiltration/ultrafiltration process is occurred twice in present invention. The so-called tangential flow refers to a form of filtration in which the liquid flow direction is perpendicular to the filtration direction. During tangential flow filtration, the flow direction of the liquid to be filtered is parallel to the direction of the filter membrane, and the liquid is passing through the membrane pores perpendicular to the membrane. Tangential flow will generate turbulence (secondary flow). Due to turbulence, the liquid flow generates shear force on the surface of filter medium (i.e., the surface of ultrafiltration membrane), which reduces the accumulation of filter cake layer or gel layer on the membrane surface, causing the precipitate to peel off from the membrane surface, reducing membrane fouling and ensuring a stable filtration speed. However, the shear force of tangential flow may disrupt the envelope structure of retrovirus, so parameters such as flow rate, membrane area, and membrane pore size need to be optimized to control the damage of shear force to retroviral activity. The present invention uses a large pore size mPES filter membrane (relative molecular mass cutoff of 750 kDa) and optimizes the shear rate to ensure permeability flux and filtration effect while reducing shear force damage, thereby reducing damage to the envelope structure of retrovirus. The overall recovery rate of retrovirus exceeded 80%. 3. The retrovirus (retroviral vector) has the characteristics of large particles and easy sedimentation, and the present invention innovatively used a combination of low-temperature, low speed centrifugation and ultrafiltration to remove more than 90% of BSA and HCP impurities in the supernatant, which also plays a role in concentrating virus solution. present invention ensures the purity of retrovirus while the concentrated virus titer reaches 10⁷ IP/ml, which meets the downstream process requirements. The present invention successfully avoids the use of ion exchange chromatography, as high salt/osmotic pressure may adversely affect the infectivity of the envelope. It also avoids the time-consuming and expensive ultracentrifugation steps, making it easy to scale up.

### DETAILED DESCRIPTION OF THE INVENTION

The present invention is described in further detail below in conjunction with specific embodiments. The examples given are only for illustrating the present invention and are not intended to limit the scope of the present invention. The examples provided below serve as examples for this technical field. It is a guide for those of ordinary skill to make further improvements and does not constitute a limitation of the invention in any way.

The experimental methods in the following examples, unless otherwise specified, are all conventional methods and are performed in accordance with the techniques or conditions described in literature in the field or in accordance with product instructions. The experimental methods in the following examples are all repeated three times unless otherwise specified.

Materials, reagents, etc. used in the following examples can all be obtained from commercial sources unless otherwise specified.

### Example 1. Preparation and Purification of Cell Supernatant Containing Recombinant Retrovirus (Retroviral Vector)

### 1. Preparation of Cell Culture Supernatant Containing a Recombinant Retrovirus (Retroviral Vector)

The cell culture supernatant of the recombinant retrovirus (retroviral vector) may be a product that has already been obtained. This example is only an example of the preparation of cell culture supernatant containing the recombinant retrovirus (retroviral vector). Preparation Method: the target gene was inserted into the retroviral expression plasmid to obtain a recombinant retroviral expression plasmid; the recombinant retroviral expression plasmid was transfected into a packaging cell line, and packaged to obtain cell culture medium containing the recombinant retrovirus (retroviral vector). The cell culture medium containing recombinant retrovirus (retroviral vector) was used to transfect the stably transfected cell line, and a stably transduced cell line that stably packages retrovirus (retroviral vector) is obtained after several subclonings; the above-mentioned stably transduced cell lines were cultivated and cell supernatants were collected to obtain cell culture supernatants containing recombinant retrovirus (retroviral vector) with stable titers.

In this embodiment, the green fluorescent protein gene EGFP was taken as an example. The EGFP gene (MK387175.1, SYN 12 Aug. 2019) was inserted between the XhoI and EcoRI recognition sites of the retroviral expression plasmid (pMSCVneo, Youbio), and kept the other sequences of pMSCVneo unchanged to obtain the recombinant retroviral expression pMSCVneo-EGFP. This pMSCVneo-EGFP was packaged to obtain recombinant retrovirus (retroviral vector), named EGFP-RV according to the following method.

### S1. Culture of Packaging Cells

6×10⁶ Phoenix Ecotropic cells (ECO, ATCC CRL-3214, less than 20 generations, not overgrown) and 10 ml of DMEM culture medium were added to each 10 cm cell culture dish, mixed thoroughly and cultured at 37° C. overnight.

### S2. Transfection of Packaging Cells

Transfection was carried out when the confluency of ECO cells reaches about 50% to 60%; add 12.5 μg of target plasmid pMSCVneo-EGFP, 250 μL of 1.25M CaCl₂), and 1 mL of H₂O into a tube for a total volume of 1.25 mL; an equal volume of 2×HBS solution to the plasmid complex was added in another tube. The plasmid complex was added into the 2×HBS solution, and vortexed for 20 s to obtain a mixture. The mixture was gently added along the edge to the ECO cell culture dish, incubated at 37° C. for 6 h, the medium was removed, and preheated fresh DMEM medium was re-added.

### S3. Obtaining Crude Virus Solution

After 48 h of transfection, the supernatant was added to obtain the virus solution, which was stored at −80° C. in aliquots. This crude virus solution was named EGFP-RV-C1.

### S4. Establishment of Virus-Producing Cell Strains

PG13 cells (ATCC, CRL-10686) was infected with EGFP-RV-C1 obtained in the above step. Two days after infection, EGFP-positive cells were enriched with EGFP antibody (Biolegend) to obtain enriched cells. A part of the enriched cells was taken to detect the expression efficiency of EGFP by flow cytometry. Another part of the enriched cells was taken to dilute into single cells and spread into 96-well plates. The supernatant on the fifth day after culturing in 96-well plates was used as the retrovirus (retroviral vector) liquid. In order to measure the viral titer of the above-mentioned retrovirus (retroviral vector) liquid by flow cytometry, retrovirus (retroviral vector) liquid was further used to infect HT1080 cells (ATCC, CCL-121). Three PG13 cell lines with the highest viral titers in 96-well plate were screened out and inoculated into 24-well plate for further culture and secondary screening. The PG13 cell supernatant on the fifth day after culture was continually used as retrovirus solution to infect HT1080 cells. The viral titer was measured by flow cytometry. The PG13 cell with the highest viral titer in cell culture supernatant was selected as the stable production strain, and stored in liquid nitrogen for long-term storage. This cell line was expanded and cultured in DMEM complete medium (DMEM medium+10% FBS+100 U/mL penicillin+10 μg/mL streptomycin+2 mM Glutamine), and cultured in a constant temperature incubator at 37° C. and 5% CO₂. The cell culture supernatant containing retrovirus was obtained, and the supernatant was named EGFP-RV-C2 as the feed liquid for subsequent purification.

### 2. Purification of a Retrovirus (Retroviral Vector)

### 2.1. Microfiltration

2.1.1 Pretreatment: the microfiltration clarification and purification system (Ripley Gold, S02-E65U-07-N) was assembled according to the assembly requirements. In this system, an intact hollow fiber column (membrane material is modified polyethersulfone mPES) with a membrane separation pore size (micromembrane pore size) of 0.65 μm and a fiber inner diameter of 0.75 mm was used, and silicone tube was used for system connection. The integrity of the system was tested by the pressure holding method and sterilized using 0.5 M or 1 M NaOH solution cycling for 30-60 min at a shear rate of 2000 s⁻¹ to 8000 s⁻¹. After sterilization, the system was emptied and filled with sterile distilled water, cycling at 6000/s for 20 to 30 min until the pH in the purification system was ≤7.0.

2.1.2 Re-equilibration: The system was emptied after washing with pure water, sterilized 0.1 M or 0.2 M PBS buffer pre-cooling at 4° C. was injected, and the system was circulated at 6000/s for 20 min.

2.1.3 Microfiltration: the microfiltration system was emptied after system balance, and the EGFP-RV-C2 cell culture supernatant containing retrovirus (retroviral vector) from step 1 was injected into the system with the flow rate of EGFP-RV-C2 to 89 mL·min⁻¹ and the shear rate to 6000 s⁻¹. The permeation end of the system was closed first to allow the virus solution to circulate in the system for 2-10 min to stabilize the system. The switch was turned on to start microfiltration and the permeate was collected as microfiltrated virus solution, named EGFP-RV-C3.

The above steps were carried out in an ice box, keeping the liquid temperature at 2-8° C.

### 2.2. Nuclease Treatment

SuperNuclease (Sino Biological) and Mg²⁺ ions were added to the EGFP-RV-C3 micro-filtered virus solution obtained in step 2.1, so that the content of nuclease was 100 U·mL⁻¹, and the concentration of Mg²⁺ ions was 2 mM, and the reaction was carried out at 4° C. for 24 h, to obtain an enzyme-digested virus solution named EGFP-RV-C4.

### 2.3. Ultrafiltration and Concentration

The ultrafiltration concentration system was used to perform ultrafiltration and concentration of the enzyme-digested virus solution obtained in step 2.2.

2.3.1 Pretreatment: the ultrafiltration purification system (Repligen, KrosFlo Research 2i) was assembled according to the assembly requirements. In this system, an intact hollow fiber column with a relative molecular mass cutoff of 750 KD, an inner diameter of the hollow fiber capillary (referred to as the fiber inner diameter) of 0.5 mm, and a semipermeable membrane of 0.16 m² was used, and silicone tube was used for the system connection.

The integrity of the system was tested by the pressure holding method and sterilized using 0.5 M or 1 M NaOH solution cycling for 30-60 min at a shear rate of 1 2000 s⁻¹ to 8000 s⁻¹. After sterilization, the system was emptied and filled with sterile distilled water, cycling at 6000/s for 20 to 30 min until the pH in the purification system was ≤7.0.

2.3.2 Re-equilibration: The system was emptied after washing with pure water, sterilized 0.1 M or 0.2 M PBS buffer pre-cooling at 4° C. was injected, and the system was circulated at 6000/s for 20 min.

2.3.3 Ultrafiltration and concentration of virus solution: the microfiltration system was emptied after system balance, and 2 L of the enzyme-digested virus solution EGFP-RV-C4 from step 2.2 was injected into the system with the flow rate of EGFP-RV-C4 to 370 mL·min⁻¹ and the shear rate to 2000 s⁻¹. The permeation end of the system was closed to allow the virus solution to circulate in the system for 2-10 min to stabilize the system. The switch was turned on to start ultrafiltration. First, the virus solution was concentrated to 1/5, then PBS buffer was continuously injected into the system to adjust the flow rate equal to the permeate flow rate of 370 mL·min⁻¹, and the volume of the virus solution was kept unchanged. Collect the retention solution, according to the 1:1 (V/V) retention volume of PBS, turn on the circulation pump to circulate the washing for 5 min, collect the first washing solution, according to the 1:1 (V/V) retention volume of PBS, turn on the circulation pump to circulate the washing for 5 min, collect the second washing solution. The above method was followed for multiple cycles, up to the third, fourth, and fifth cycles, and mix all the washing liquids collected each time together to form the primary virus concentrate, that is, the primary ultrafiltered virus solution, named EGFP-RV-C5(5). The volume of EGFP-RV-C5 (5) obtained in this step was 400 mL.

2.3.4 The virus solution is concentrated again by ultrafiltration

Due to the large volume of the actual ultrafiltration purification starting material, an additional ultrafiltration concentration step was required. Before the above-mentioned ultrafiltration step, the present invention added a large-volume hollow fiber column (membrane area: 0.16 m², other parameters are the same as above) to concentrate the starting material liquid by 5 times. Replacing the hollow fiber column of the above-mentioned ultrafiltration purification system, the new hollow fiber column had a relative molecular mass of 750 KD, an inner diameter of the hollow fiber capillary (referred to as the fiber inner diameter) of 0.5 mm, and a membrane area of the semipermeable membrane of 0.0115 m². Sterilization and balancing were performed as above, 400 mL EGFP-RV-C5 (5) was injected into the system with the flow rate of EGFP-RV-C2 (5) to 53 mL·min⁻¹ and the shear rate to 2000 s⁻¹. The permeation end of the system was closed to allow the virus solution to circulate in the system for 2-10 min to stabilize the system. The switch was turned on to start ultrafiltration. First, the virus solution was concentrated to 1/10, then PBS buffer was continuously injected into the system to adjust the flow rate equal to the permeate flow rate of 53 mL·min⁻¹, and the volume of the virus solution was kept unchanged. Collect the retention solution, according to the 1:1 (V/V) retention volume of PBS, turn on the circulation pump to circulate the washing for 5 min, collect the washing solution. The washing liquid and retention solution were mixed to form the secondary virus concentrate, that is, the ultrafiltered virus solution, named EGFP-RV-C6. The volume of EGFP-RV-C6 obtained in this step was 400 mL.

### 2.4. Low Speed Centrifugation and Sterilization Filtration

The 40 mL ultrafiltered virus solution EGFP-RV-C6 obtained in step 2.3 was further centrifuged at low speed using a centrifugal force of 6000 g at 4° C. for 16 h. After centrifugation, the supernatant was discarded and the precipitate was collected, which was the unsterilized virus. The precipitate was resuspended with PBS containing 2% human albumin in a volume of 1/10 of that before centrifugation at 4° C. for 1 h to obtain a virus resuspension named EGFP-RV-C7.

Finally, the virus suspension EGFP-RV-C7 was filtered and sterilized using a PVDF membrane filter with a pore size of 0.22 μm to obtain a purified virus solution, which was a GMP-grade retrovirus with a volume of 4 mL named EGFP-RV-F and placed at −80° C.

### 3. Biological Trait Detection of Retrovirus EGFP-RV-F

### 3.1. Titer Detection of Retrovirus

The number of HT1080 cells infected by per unit volume retrovirus, i.e., the viral activity titer or the number of infectious particles (Infectious particles, IP/mL), was indirectly determined by the expression of EGFP using a flow-through assay.

First, HT1080 cells in the logarithmic phase of growth were collected and digested with trypsin for 2 to 3 min, and complete culture medium was added to prepare a cell suspension. HT1080 cells in the logarithmic growth phase were inoculated at a density of 1.5×10⁵ cells/well. After the cells were spread on the TC6-well plate, they were shaken up and down, marked, and placed in a carbon dioxide incubator at 37° C. for 20 to 24 h. During transduction, virus solution containing 8 μg/mL polybrene was added to each well, in which the virus solution and complete medium were diluted in different proportions. After transduction, the cells were incubated in a CO₂ incubator at 37° C. for 1 h, shaking every 15 min. After the fourth shake, 2 mL of complete medium was added, and the incubation was continued for 48 h. The viral transduction solution in the wells was aspirated, and 2 mL PBS was added to each well to rinse the cells. 100 μL of 0.25% trypsin was added to each well and place at room temperature for 1 to 2 min. 500 μL of complete medium was added to each well to terminate digestion. A pipette tip was used to pipette the cell suspension, centrifuge at 1500 rpm for 5 min, and the cell pellet was collected.

The cell suspension was inoculated into 96-well plates at 3×10⁴ cells/well, centrifuged at 1500 rpm for 5 min. Each well was washed once with 200 μL FACS buffer, centrifuged at 1500 rpm for 5 min. 20 μL FITC-labeled EGFP antibody (Biolegend) was added to each well at a dilution of 1:100 and incubate in the dark for 10 min. Centrifugation was performed again and 200 μL FACS buffer was added to each well. Flow cytometry analysis was performed on the machine.

The viral titer was calculated according to the following formula:

Viral titer (IP·mL⁻¹)=(F×N)/V
F: cell positivity rate detected by flow cytometry (measured value−blank control CTR value)
N: Number of cells (3×10⁵)/well at transfection
V: mL of virus added
Virus recovery rate: harvested viral titer/initial viral titer×100%.

### 3.2. Detection of Residual Host Cell DNA (HCD)

Pretreatment of samples: residual host cell DNA sample preparation kit (paramagnetic particle method) (Huzhou Shenke, SK030203D100) was used.

qPCR detection of samples was performed according to the following steps:

a. Preparation of PG13 DNA quantitative reference and standard curve:

PG13 cells were collected, and genomic DNA was extracted using AllPrep DNA/RNA Mini Kit (QIAGEN, 80204) as a quantitative reference. Use 1×PBS (pH 7.4, without Ca and Mg) to perform gradient dilution of the DNA quantification reference. The dilution concentrations were 3 ng/μl, 300 pg/μL, 30 pg/μL, 3 pg/μL, 300 fg/μL, 30 fg/μL, and 3 fg/μL.

Seven clean 1.5 ml centrifuge tubes were taken and labeled as ST0, ST1, ST2, ST3, ST4, ST5, and ST6 respectively. The DNA quantification reference was diluted to 3 ng/μL with PBS in the ST0 tube, shaked and mixed well, and then centrifuged quickly for 10 seconds. 90 μL PBS was added to ST1, ST2, ST3, ST4, ST5, and ST6 tubes respectively. Gradient dilutions was done sequentially.

Set up the recovery quality control ERC: the sample prepared with 300 pg quantitative reference material was used as the ERC, and 100 μl of the sample to be tested was added to a 1.5 ml clean centrifuge tube. Another 10 μL of ST2 was added, mixed well, and labeled as sample ERC.

Set negative quality control NCS: add 100 μL of sample matrix solution (or DNA diluent) into a 1.5 ml clean centrifuge tube and label it as negative quality control NCS.

b. Preparation of qPCR reaction solution (qPCR MIX)

The number of reaction wells required was calculated based on the number of standard curve and samples to be tested. Generally, 3 replicate will be made for each well/sample. Number of reaction wells=(standard curves of 6 concentration gradients+1 control NTC without template+1 negative quality control NCS+sample to be tested×2)×3; the total amount of qPCR MIX required was calculated based on the number of reaction wells, and the qPCR MIX was prepared according to the formula in Table 1. The primer sequences are as follows:

Probe,
5′-FAM-AGGGCCCCCAATGGAGGAGCT-TAM-3′;
Forward primer,
5′-CCCCTTCAGCTCCTTGGGTA-3′;
and
Reverse primer,
5′-GCCTGGCAAATACAGAAGTGG-3′.

Q-PCR probe mix was purchased as a ready-made commercial premix (Vazyme, AceQ PCR probe master mix).

c. qPCR reaction: each reagent in Table 2 was mixed well on ice. The total volume was 25 μL after adding samples. Establish qPCR template and running parameters: 95° C., 10 min, 1 cycle; 95° C., 15 s, 60° C., 1 min, 40 cycle; 40° C., 30 s, 1 cycle.

d. detection and result analysis: the program was run on the ABI 7500 qPCR instrument. In the Report panel of Results, the detection values of the non-template control NTC, negative quality control NCS, sample to be tested, and sample ERC may be read in pg/10 μL on the Mean Quantity column. Subsequently, the unit can be converted to pg/μL or pg/mL in the test report. The sample recovery rate was calculated based on the test results of samples to be tested and the sample ERC and required to be between 50% and 150%. The results of NTC should be Undetermined or Ct value ≥35. The Ct value of NCS should be greater than the lowest concentration of Ct value of the standard curve.

TABLE 1
qPCR MIX Preparation

| Components | Single-well Volume (μL) |
|---|---|
| qPCR probe mix | 12.5 |
| mDNA-F | 2 |
| mDNA-R | 2 |
| mDNA-P | 1 |
| RNAse-free water | Complement to |
| | reaction volume |
| Total | 20 |

TABLE 2
Sample preparation

| | |
|---|---|
| standard curve | 20 μL qPCR MIX + 5 μL ST1/ST2/ST3/ST4/ |
| | ST5/ST6 |
| NTC | 20 μL qPCR MIX + 5 μL DNA diluent |
| | solution |
| negative control | 20 μL qPCR MIX + 5 μL negative quality |
| | control NCS |
| Sample to be tested | 20 μL qPCR MIX + 5 μL sample to be tested |
| ERC | 20 μL qPCR MIX + 5 μL sample ERC |

### 3.3. Detection of Residual Bovine Serum Albumin (BSA)

Detection method of BSA is as follows: Bovine Serum Albumin (BSA) ELISA kit (Cygnus, F030) was used to detect the BSA residues in the samples. This kit provided an anti-BSA monoclonal antibody-coated plate, and enzyme-labeled anti-BSA polyclonal antibodies were used as detection antibodies to form this double-antibody sandwich ELISA detection kit. According to the supplier's instructions, the sample or standard to be tested was added first, so that the BSA therein binded to the antibodies immobilized on the plate. After thorough washing, HRP-labeled anti-BSA polyantibody was added and incubated. After washing, TMB substrate was added, and TMB was converted into blue under the catalysis of HRP, and finally converted into yellow under the action of termination solution. The shade of color is related to the amount of BSA in the sample or standard. Finally, a microplate reader was used to measure the absorbance value of each sample well at 450 nm, and the BSA concentration in the sample to be tested was calculated by the BSA standard curve.

### 3.4. Detection of Residual Host Cell Protein (HCP)

The present invention uses the ELISA sandwich method to detect HCP in the sample. HCP antibodies are polyclonal antibodies purified from the serum of rabbits immunized with PG13 cell culture supernatant protein. The specific detection method is as follows:

a. Plate coating: anti-PG13 cell supernatant protein antibody was coated on the surface of the reaction well of test plate at the optimal concentration. The optimal antibody concentration was determined by drawing a standard curve using PG13 cell supernatant proteins of known concentrations that had the required sensitivity and precision within the required effective concentration range. For PG13 cell supernatant protein, the effective concentration range that this kit can detect is 62.5 ng/mL to 4000 ng/mL. Those skilled in the art can easily determine whether appropriate sensitivity and accuracy are present in the required range without performing unnecessary experiments.

b. Plate washing: the coating solution was poured off, wash buffer (approximately 400 μl per well) was added and poured off. Repeat this wash cycle as many times as required. The washing buffer can be 0.01 mol/L phosphoric acid buffer (0.0027 mol/L potassium chloride, 0.137 mol/L sodium oxide, pH 7.4, containing 0.01% w/v of TritonX-100).

c. Plate blocking: protein and detergent blocking buffer (coating buffer solution containing 1% BSA/0.1% Triton X-100) was added to the reaction wells. Plates can be stored in this form.

d. Addition of samples and standards: the plate was washed as described above. 100 μl standard and 100 μl sample to be tested were added into the reaction wells, then 50 μl conjugate reagent was added to each well, mixed gently for 15 seconds and incubated at 37° C. for 60 min. The reaction solution was discarded and the reaction plate was washed with buffer for 5 times. With excess water absorbed, 50 μL of chromogenic solution was added to the reaction well, and incubated at 37° C. for 15 min to terminate the reaction. The reaction plate was placed on a microplate reader to read the optical density value.

e. TMBS was used as the chromogenic substrate and the absorbance was read at 450 nm. The accurate concentration of HCP can be calculated by reading the absorbance of the test sample and then referring to the standard curve made by the HCP standards.

### 3.5. Detection of Residual Supernuclease

Nuclease can completely digest RNA and DNA with at least 5 phosphate residues (single-stranded, double-stranded, linear, circular and super-helical) to form 5′-monophosphate-terminal oligonucleotides with a length of 3-5 bases. The present invention uses nuclease to degrade HCD in the process so that it can be easily removed by subsequent purification methods. However, since nuclease itself is also a protein impurity, it needs to be removed together with other impurities in the subsequent purification process. The present invention detects the residual amount of nuclease in downstream products by SuperNuclease ELISA kit (Sino Biological). For specific testing steps, please refer to Sino Biological's official website and product manual.

Repeat 3 times according to the method described in the purification of retrovirus (retroviral vector) in step 2 of Example 1 to obtain 3 batches of GMP-grade retrovirus (retroviral vector) EGFP-RV-F, which are named batch 1, batch 2 and batch 3 respectively. Detect the content of nuclease, BSA, HCP, HCD and the viral titer of batch 1, batch 2 and batch 3 according to the above method.

The results showed that after the above steps of microfiltration, nuclease treatment, ultrafiltration and low speed centrifugation, the concentration of the liquid volume is 500 times. The yield of GMP-grade retrovirus EGFP-RV-F is between 10-20% with activity titer>1.0E+07, HCD<100 ng/ml, nuclease below the detection limit (<3.15 ng/ml), HCP<1 μg/mL, and BSA<200 ng/mL, which meets the requirements of downstream production requirements (Table 9).

TABLE 9
Impurity residues and overall recovery rate of final
purified products of a retrovirus (retroviral vector)

| | BSA | Nuclease | HCP | HCD | Starting material | Purified | Recovery |
|---|---|---|---|---|---|---|---|
| GMP-grade | residual | residual | residual | residual | activity titer | activity titer | rate |
| retrovirus | (ng/mL) | (ng/mL) | (ng/mL) | (ng/mL) | (IP · mL⁻¹) [1] | (IP · mL⁻¹)[2] | (%) |
| Batch 1 | 198 | below the | 486 | 87 | 8.86E+05 | 8.22E+07 | 18.56 |
| | | detection | | | | | |
| | | limit | | | | | |
| Batch 2 | 142 | below the | 315 | 64 | 4.32E+05 | 3.54E+07 | 16.42 |
| | | detection | | | | | |
| | | limit | | | | | |
| Batch 3 | 146 | below the | 330 | 53 | 6.84E+05 | 5.07E+07 | 14.82 |
| | | detection | | | | | |
| | | limit | | | | | |

Note:
[1] Starting material activity titer refers to the titer converted into a volume of 2 L of material liquid;
[2]Purified activity titer refers to the titer converted into a volume of 4 mL of feed solution.

### Example 2. Optimization of Purification Process of Retrovirus (Retrovirus Vector)

### 2.1. Optimization of Microfiltration Process of Crude Virus Solution

Referring to 2.1 Microfiltration in Step 2 of Example 1, the recovery rates of microfiltration was obtained by using different flow rates and membrane pore sizes to control the shear force.

The operation was the same as step 2.1 in Example 1 except that the flow rate of EGFP-RV-C2 (the titer is 8.86×10⁵ IP mL⁻¹) in step 2.1.3 of Example 1 was changed from 89 mL·min⁻¹ to 133.5 mL·min⁻¹ and 44.5 mL·min⁻¹ respectively, the shear rate was changed from 6000 s⁻¹ to 1000 s⁻¹ and 2000 s⁻¹ respectively, and the micromembrane pore size was replaced from 0.45 μm to 0.65 μm.

The results are shown in Table 3. It can be seen that the optimal recovery rate of retrovirus could be obtained when the flow rate is 89 mL·min⁻¹, the shear force is controlled at 2000 s⁻¹, and a membrane pore size is 0.65 μm.

TABLE 3
Effects of different microfiltration conditions on virus recovery rate

| | | | Micro- | | |
|---|---|---|---|---|---|
| | Flow rate | Shear | membrane | Titer | Recovery |
| | (mL · | rate | pore size | (IP · | rate |
| Materials | min⁻¹) | (s⁻¹) | (μm) | mL⁻¹) | (%) |
| EGFP-RV-C2 | — | — | — | 8.86E+05 | 100 |
| EGFP-RV-C3 | 133.5 | 3000 | 0.65 | 7.80E+05 | 88.04 |
| EGFP-RV-C3 | 89 | 2000 | 0.65 | 8.71E+05 | 98.31 |
| EGFP-RV-C3 | 44.5 | 1000 | 0.65 | 5.71E+05 | 64.44 |
| EGFP-RV-C3 | 133.5 | 3000 | 0.45 | 6.52E+05 | 73.59 |
| EGFP-RV-C3 | 89 | 2000 | 0.45 | 7.68E+05 | 86.68 |
| EGFP-RV-C3 | 44.5 | 1000 | 0.45 | 5.21E+05 | 58.80 |

### 2.2. Optimization of Nuclease Treatment

Referring to the nuclease treatment in step 2.2 of Example 1, different nuclease concentrations, treatment times and temperatures were used to obtain the recovery rate after nuclease treatment under different conditions.

The operation was the same as step 2.2 in Example 1 except that the nuclease content was changed from 100 U·mL⁻¹ to 50 U·mL⁻¹ and 25 U·mL⁻¹ respectively, the reaction time was changed from 24 h to 8 h and 16 h respectively.

The results are shown in Table 4. It can be seen that the highest HCD removal efficiency could be obtained when the enzyme concentration is 100 U·mL⁻¹ and treated at 4° C. for 24 h.

TABLE 4
Removal efficiency of nuclease treatment
on HCD under different conditions

| | Enzymatic | | | HCD con- | |
|---|---|---|---|---|---|
| | activity | | Temper- | centration | Removal |
| | (U · | Time | ature | (ng · | rate |
| Materials | mL⁻¹) | (h) | (° C.) | mL⁻¹) | (%) |
| EGFP-RV-C3 | — | — | — | 2292 | 0 |
| EGFP-RV-C4 | 100 | 8 | 4 | 89.68 | 96.09 |
| EGFP-RV-C4 | 50 | 8 | 4 | 137.95 | 93.98 |
| EGFP-RV-C4 | 25 | 8 | 4 | 180.49 | 92.13 |
| EGFP-RV-C4 | 100 | 16 | 4 | 76.54 | 96.67 |
| EGFP-RV-C4 | 50 | 16 | 4 | 103.79 | 95.47 |
| EGFP-RV-C4 | 25 | 16 | 4 | 146.69 | 93.60 |
| EGFP-RV-C4 | 100 | 24 | 4 | 53.72 | 97.66 |
| EGFP-RV-C4 | 50 | 24 | 4 | 74.95 | 96.73 |
| EGFP-RV-C4 | 25 | 24 | 4 | 155.35 | 93.22 |
| EGFP-RV-C4 | 100 | 8 | 37 | 165.7 | 92.77 |
| EGFP-RV-C4 | 50 | 8 | 37 | 185.47 | 91.91 |
| EGFP-RV-C4 | 25 | 8 | 37 | 260.37 | 88.64 |
| EGFP-RV-C4 | 100 | 16 | 37 | 135.63 | 94.08 |
| EGFP-RV-C4 | 50 | 16 | 37 | 169.23 | 92.62 |
| EGFP-RV-C4 | 25 | 16 | 37 | 181.03 | 92.10 |
| EGFP-RV-C4 | 100 | 24 | 37 | 127.56 | 94.43 |
| EGFP-RV-C4 | 50 | 24 | 37 | 211.67 | 90.76 |
| EGFP-RV-C4 | 25 | 24 | 37 | 266.52 | 88.37 |

### 2.3. Optimization of Ultrafiltration Process

Different relative molecular weight cutoffs of hollow fiber membranes will have different effects on the recovery rate and impurity removal rate. The larger the molecular weight cutoff of the hollow fiber, the higher the impurity removal rate. However, if the molecular weight cutoff exceeds the size of the virus particles, it may also cause the loss of some virus particles and affect the recovery rate.

Referring to the ultrafiltration process in step 2.3 in Example 1, hollow fiber columns with different molecular weight cutoffs were used to obtain the effects of hollow fiber membranes with different molecular weight cutoffs on the recovery rate and impurity removal rate.

The relative molecular mass cutoff of the hollow fiber membrane in step 2.3 in Example 1 was changed to 750 kDa, 500 kDa, and 300 kDa, keeping other conditions unchanged, and the results are shown in Table 5.

TABLE 5
Effects of different relative molecular weight cutoffs of hollow
fiber membranes on virus recovery rate and impurity removal rate

| | Relative | | | | | | |
|---|---|---|---|---|---|---|---|
| | molecular | Nuclease | HCP | BSA | HCD | | |
| | weight | removal | Removal | Removal | Removal | | Recovery |
| | cutoffs | rate | rate | rate | rate | Titer | rate |
| Materials | (kDa) | (%) | (%) | (%) | (%)[1] | (IP · mL⁻¹) | (%) |
| EGFP-RV-C3 | — | — | — | — | — | 7.61E+05 | 100 |
| EGFP-RV-C6 | 300 | 98.15 | 98.70 | 99.49 | 88.16 | 5.78E+05 | 75.95 |
| EGFP-RV-C6 | 500 | 98.89 | 98.62 | 99.48 | 96.20 | 4.59E+05 | 60.32 |
| EGFP-RV-C6 | 750 | 99.22 | 99.15 | 99.63 | 97.92 | 6.16E+05 | 80.95 |

Note:
[1]The calculation of HCD removal rate is compared with the feed solution before nuclease treatment, i.e., HCD (EGFP-RV-C6)/HCD (EGFP-RV-C3) × 100%

After determining the relative molecular mass cutoff of the hollow fiber membrane, the effects of different shear rates and fiber inner diameters on the recovery rate of the virus solution were further explored. Since retrovirus (retroviral vector) particles are sensitive to shear force, the shear rate will directly affect the virus activity and recovery rate. In addition, the inner diameter of hollow fiber tube will directly affect the material liquid flux. For the same volume of material solution, the smaller the inner diameter, the smaller the flux and the slower the flow rate.

Referring to the ultrafiltration process of step 2.3 in Example 1, the flow rate of the ultrafiltered virus was controlled at 38, 53, 79, 100, 106, 141, 212, 283 mL·min⁻¹, the shear rate was controlled at 1441, 2000, 3000, 4000 s⁻¹, the fiber inner diameter was selected as 0.5 and 1.0 mm, and the membrane area was selected as 75 and 115 cm², keeping other processes in 2.3 unchanged, and the results are shown in Table 6.

The results of Table 6 showed that using a hollow fiber column with the inner diameter of 0.5 mm, membrane area of 115 cm², liquid flow rate of 53 mL·min⁻¹, and shear rate at 2000 s⁻¹, the maximum virus recovery rate can be obtained.

TABLE 6
Effects of different shear rates and fiber inner diameters on virus recovery rate

| | | Fiber | | | | |
|---|---|---|---|---|---|---|
| | | inner | Area of | Shear | | Recovery |
| | Flow rate | diameter | membrane | rate | Titer | rate |
| Materials | (mL · min⁻¹) | (mm) | (cm²) | (s⁻¹)[1] | (IP · mL⁻¹) | (%) |
| EGFP-RV-C4 | | | | — | 5.40E+05 | 100 |
| EGFP-RV-C6 | 100 | 1.0 | 75 | 1441 | 3.57E+05 | 66.11 |
| EGFP-RV-C6 | 141 | 1.0 | 75 | 2000 | 3.24E+05 | 60.00 |
| EGFP-RV-C6 | 212 | 1.0 | 75 | 3000 | 3.02E+05 | 55.93 |
| EGFP-RV-C6 | 283 | 1.0 | 75 | 4000 | 3.08E+05 | 57.04 |
| EGFP-RV-C6 | 38 | 0.5 | 115 | 1441 | 4.48E+05 | 82.96 |
| EGFP-RV-C6 | 53 | 0.5 | 115 | 2000 | 5.32E+05 | 98.52 |
| EGFP-RV-C6 | 79 | 0.5 | 115 | 3000 | 4.19E+05 | 77.59 |
| EGFP-RV-C6 | 106 | 0.5 | 115 | 4000 | 3.68E+05 | 68.15 |

Note:
[1]This test adjusts the shear rate by changing the flow rate in a certain type of hollow fiber column.

According to the principle of ultrafiltration, a certain amount of impurities can be removed in each cycle. The more cycles, the higher the impurity removal rate. However, tangential flow generated by the ultrafiltration process can also cause the inactivation of a part of virus. Therefore, the other steps of ultrafiltration process in step 2.3 of the example were kept unchanged and the effects of different cycle times on virus recovery rate were tested (Table 7). The results showed that circulating the ultrafiltration 5 times improved the removal efficiency of some impurities while ensuring the recovery rate to the greatest extent.

TABLE 7
Effects of different cycles of ultrafiltration and concentration
on virus recovery rate and impurity removal rate ^{[1]}

| | | | Recov- | BSA | BSA |
|---|---|---|---|---|---|
| | Number | Titer | ery | concen- | Removal |
| | of | (IP · | rate | tration | rate |
| Materials | Cycles | mL⁻¹) | (%) | (ng/mL) | (%) ^{[2]} |
| EGFP-RV-C4 | — | 5.78E+05 | 100 | 2978000 | — |
| EGFP-RV-C5(1) | 1 | 1.36E+06 | 94.3 | 982740 | 86.8 |
| EGFP-RV-C5(2) | 2 | 1.35E+06 | 93.8 | 550930 | 92.6 |
| EGFP-RV-C5(3) | 3 | 1.34E+06 | 92.7 | 461590 | 93.8 |
| EGFP-RV-C5(4) | 4 | 1.32E+06 | 91.3 | 335025 | 95.5 |
| EGFP-RV-C5(5) | 5 | 1.30E+06 | 90.0 | 91919 | 98.8 |
| EGFP-RV-C5(6) | 6 | 1.02E+06 | 70.6 | 84561 | 98.9 |
| EGFP-RV-C5(7) | 7 | 9.40E+05 | 65.1 | 69779 | 99.1 |
| EGFP-RV-C5(8) | 8 | 8.28E+05 | 57.3 | 49444 | 99.3 |

Note:
^{[1]} Concentrated by 2.5 times.
^{[2]} BSA removal rate = [BSA (EGFP-RV-C5) × harvest volume]/[BSA (EGFP-RV-C4) × starting volume].

### 2.4. Optimization for Removing HCP and BSA by Low Speed Centrifugation

After the ultrafiltration step, HCD was <100 ng/ml and nuclease was below the detection limit (<3.15 ng/ml), indicating that these two process residue indicators had basically met the requirements. However, HCP and BSA were still slightly higher than the standard, with median mean values of 13078 ng/ml and 2056 ng/ml respectively (EGFP-RV-C6).

Referring to the low speed centrifugation process in step 2.4 in Example 1, different centrifugal forces and centrifugation times were used, keeping other process in step 2.4 unchanged, to obtain the results of virus recovery rate and impurity removal rate under different centrifugal forces and centrifugation times (Table 8). The results showed that after centrifugation at 6000 g at 4° C. for 16 h, HCP<1 μg/mL and BSA<200 ng/ml in the purified liquid met the requirements of downstream production.

TABLE 8
Virus recovery rate and impurity removal rate after low speed centrifugation under different conditions ^{[1]}

| | | | | | | BSA | | HCP |
|---|---|---|---|---|---|---|---|---|
| | centrifugal | | | Recovery | BSA | Removal | HCP | Removal |
| | force | Centrifugal | Titer | rate | Concentration | rate | Concentration | rate |
| Materials | (g) | time (h) | (IP · mL⁻¹) | (%) | (ng/mL) | (%) ^{[2]} | (ng/mL) | (%) ^{[3]} |
| EGFP-RV-C6 | — | — | 9.10E+06 | — | 2198 | — | 13078 | — |
| EGFP-RV-C7 | 4000 | 4 | 1.03E+07 | 22.64 | 105 | 99.04 | 568 | 99.13 |
| EGFP-RV-C7 | 4000 | 8 | 2.13E+07 | 46.81 | 165 | 98.50 | 780 | 98.81 |
| EGFP-RV-C7 | 4000 | 16 | 2.22E+07 | 48.79 | 174 | 98.42 | 894 | 98.63 |
| EGFP-RV-C7 | 4000 | 24 | 1.52E+07 | 33.41 | 145 | 98.68 | 691 | 98.94 |
| EGFP-RV-C7 | 6000 | 4 | 1.23E+07 | 27.03 | 112 | 98.98 | 541 | 99.17 |
| EGFP-RV-C7 | 6000 | 8 | 2.83E+07 | 62.20 | 154 | 98.60 | 728 | 98.89 |
| EGFP-RV-C7 | 6000 | 16 | 3.12E+07 | 68.57 | 198 | 98.20 | 486 | 99.26 |
| EGFP-RV-C7 | 6000 | 24 | 2.18E+07 | 47.91 | 210 | 98.09 | 856 | 98.69 |
| EGFP-RV-C7 | 8000 | 4 | 1.64E+07 | 36.04 | 142 | 98.71 | 648 | 99.01 |
| EGFP-RV-C7 | 8000 | 8 | 2.44E+07 | 53.63 | 146 | 98.67 | 630 | 99.04 |
| EGFP-RV-C7 | 8000 | 16 | 2.22E+07 | 48.80 | 185 | 98.32 | 962 | 98.53 |
| EGFP-RV-C7 | 8000 | 24 | 1.78E+07 | 39.12 | 243 | 97.79 | 1015 | 98.45 |

Note:
^{[1]} Concentrated by 5 times.
^{[2]} BSA removal rate = [BSA (EGFP-RV-C7) × harvest volume]/[BSA (EGFP-RV-C6) × starting volume].
^{[3]} HCP removal rate = [HCP (EGFP-RV-C7) × harvest volume]/[HCP (EGFP-RV-C6) × starting volume].

The present invention has been described in detail above. For those skilled in the art, the present invention can be implemented within a wide range parameters, concentrations and conditions without departing from the spirit and scope of the invention and without unnecessary experiments. Although specific embodiments of the present invention have been shown, it should be understood that further improvements can be made to the invention. In short, based on the principles of the present invention, this application is intended to include any change, use, or improvement of the present invention, including changes that depart from the scope disclosed in this application and are made using conventional techniques known in the art. Some essential features can be carried out according the scope of the appended claims below.

1-17. (canceled)

18. A method for purifying retrovirus, comprising the following steps:
A), performing a microfiltration of a cell culture supernatant containing a retrovirus to remove cell debris in the cell culture supernatant to obtain a microfiltrated virus solution;
B), performing a treatment on the micro-filtered virus solution with a nuclease to degrade host DNA residues into small DNA fragments to obtain an enzyme-digested virus solution;
C), performing an ultrafiltration on the enzyme-digested virus solution so that the retrovirus is remained in a retentate, collect the retentate, and obtain an ultrafiltered virus solution; and
D), performing a low speed centrifugation for the ultrafiltered virus solution to precipitate the retrovirus, and collecting the precipitate to obtain unsterilized virus.

19. The method of claim 18, further comprising a step of filtering and sterilizing the unsterilized virus to obtain purified retrovirus.

20. The method according to claim 18, wherein, in the microfiltration, a membrane pore size (micromembrane pore size) of the semipermeable membrane used is 0.45 μm-0.75 μm, and/or the membrane material of the semipermeable membrane used is modified polyethersulfone.

21. The method of claim 18, wherein, in the nuclease treatment, the nuclease in the concentration in the reaction system is 1 U·mL⁻¹ to 500 U·mL⁻¹, and/or the reaction is performed at 2° ° C. to 8° ° C. for 8 h to 24 h.

22. The method according to claim 18, wherein, in the ultrafiltration in step C), a relative molecular weight cutoff of the ultrafiltration is 350 KD to 750 KD or 750 KD.

23. The method according to claim 18, wherein, the ultrafiltration in step C) comprises:
c1), using a hollow fiber column with a relative molecular mass cutoff of 750 KD, an inner diameter of the hollow fiber capillary of 0.5 mm, and a semipermeable membrane area of 0.16 m² to perform an ultrafiltration with a shear rate of 2000 s⁻¹ on the enzyme-digested virus solution to obtain a primary virus ultrafiltrate;
c2), for the primary virus ultrafiltrate, using a hollow fiber capillary with a relative molecular mass cutoff of 750 KD, an inner diameter of a hollow fiber column of 0.5 mm, and a semipermeable membrane area of 0.0115 m² to perform an ultrafiltration with a shear rate of 2000 s⁻¹ to obtain a secondary virus ultrafiltrate.

24. The method according to claim 18, wherein, the processing conditions of a low speed centrifugation in step D) are: a centrifugal force of 4000 g to 10000 g, and centrifugation for 4 h to 24 h.

25. The method of claim 20, wherein, in the microfiltration, a flow rate of the cell culture supernatant is 89 mL·min⁻¹ to 133.5 mL·min⁻¹, 89 mL·min⁻¹ or 133.5 mL·min⁻¹.

26. The method of claim 20, wherein, in the microfiltration, a shear rate of the microfiltration is 2000 s⁻¹ to 3000 s⁻¹, 2000 s⁻¹ or 3000 s⁻¹.

27. The method of claim 18, wherein, a temperature of the liquid is maintained at 2° C. to 8° C. during the microfiltration.

28. The method of claim 24, wherein, a temperature of low speed centrifugation is 2° ° C. to 8° C.

29. The method of claim 28, wherein, the temperature of low speed centrifugation is 4° C.

30. A retrovirus obtained by the method of claim 18.

31. The retrovirus according to claim 30, wherein, BSA of the retrovirus is <200 ng/ml, HCP is <1 μg/ml, and HCD is <100 ng/ml.

32. A product, wherein, the product contains the retrovirus according to claim 30.

33. A method for preparing a therapeutic product.

34. The method according to claim 33, wherein the method uses a retrovirus obtained by a method for purifying retrovirus, comprising the following steps:
A), performing a microfiltration of a cell culture supernatant containing a retrovirus to remove cell debris in the cell culture supernatant to obtain a microfiltrated virus solution;
B), performing a treatment on the micro-filtered virus solution with a nuclease to degrade host DNA residues into small DNA fragments to obtain an enzyme-digested virus solution;
C), performing an ultrafiltration on the enzyme-digested virus solution so that the retrovirus is remained in a retentate, collect the retentate, and obtain an ultrafiltered virus solution; and
D), performing a low speed centrifugation for the ultrafiltered virus solution to precipitate the retrovirus, and collecting the precipitate to obtain unsterilized virus.

35. The method according to claim 33, wherein the therapeutic product comprises a gene therapy product and/or a cell therapy product and/or an immunotherapy product.
